(19) United States
(12) Patent Application Publication
(10) Pub. No.: US 2017/0000652 A1
(43) Pub. Date: Jan. 5, 2017

(54) Lip and/or nose bandage

(71) Applicant: Gayla Marie Guice, Ocean Springs, MS (US)

(72) Inventor: Gayla Marie Guice, Ocean Springs, MS (US)

(21) Appl. No.: 14/545,892

(22) Filed: Jul. 2, 2015

(51) Int. Cl. A61F 13/12; A61F 13/02

(52) U.S. Cl. CPC A61F 13/122; A61F 13/0233; A61F 13/0256; A61F 13/126; A61F 2013/00314

(57) ABSTRACT

The invention is directed to a bandage, particularly a lip and/or nose bandage for use on wounds (surgical or traumatic), scratches, sores, cold sores, blisters, fever blisters, ulcers, pimples, lesions, and burns. The lip and/or nose bandage comprises a base sheet layer (on one end associated with at least one skin adhesive); a dressing layer; and either or both of: (the same base sheet layer) on the other end associated with at least one mucosal adhesive; and/or a malleable layer positioned between the base sheet and dressing layers.

### TECHNICAL FIELD

The present invention relates to wound dressings, particularly an adhesively applied bandage adapted for use on the lip and/or (alar sidewall of the) nose.

### BACKGROUND ART

The popularity of the adhesive bandage, originally patented in 1929, is not surprising as it provides many known advantages such as protection, controlled wound environment, and concealment. As described at length in U.S. Pat. No. 7,943,811, there has since been patented many types of “improved adhesive bandages” which among many advantages, promote healing by incorporating, for example, an antibiotic agent. Various forms (like those adapted for use on fingertips) and categories (like those created to optimize moisture retention/absorption), have also been invented, in some cases by adding additional layers to the composed bandage.

Still, an adhesive bandage generally comprises a first layer (base sheet) for covering the wound site and an area around the wound site and onto which an adhesive is incorporated to secure it and at least one optimally non-adhering second layer (dressing) for placement immediately over the wound and coming into contact with the wound. The dressing is smaller, by varying percentages, than the base sheet.

The application of an adhesive bandage onto the nose (mainly, the alar sidewalls) and/or lips (and that area immediately surrounding the lips) has presented a challenge to those desiring the advantages (both therapeutic and aesthetic) of covering wounds there located.

U.S. Pat. No. 4,534,342 describes a nose bandage that envelopes the entire nose. While this bandage may address coverage of a wound located on the exterior of the alar sidewall, it does not address those located on the inside and/or towards the edge of it. Further, it offers very little inconspicuousness and is most likely practical only in clinical settings.

U.S. Pat. No. 1,111,679 describes a bendable clip (of unspecified composition) that holds non-adhesive dressings in place, with this assembly being further secured by adhesive tape. The use of this combination of items, by today's standards, would prove cumbersome and inconvenient.

In the case of a conventional two-ended adhesive bandage, the inability to apply (and/or the undesired result of applying) one of the two adhesive ends onto the mucosal tissue of the inside of the lips and/or nose prevents its effective use.

In the case of non-adhesive wound dressings (such as gauze or absorbent pads) used in conjunction with separate adhesive tapes, a similar problem exists and the alternative is to wrap the entire head of the person with securing devices, which is impractical for all but clinical settings and generally an unwelcomed solution.

The American Sexual Health Association (ASHA) reports that more than 50% of American adults are infected with the herpes virus that causes cold sores or fever blisters. Currently, the options for “covering” and “protecting” these sores and blisters are limited to products called cold sore “patches” or “bandages” which are generally small, circular coverings with the entire surface of the layer next to the skin being cohesive. That is, the “patch” sticks to the sore risking re-injury upon removal.

### SUMMARY OF INVENTION

In a first aspect, the present invention solves the problem of attachment of a bandage to the lip and/or area surrounding the lip and/or (alar sidewall of the) nose by incorporating a malleable component or material into an adhesive bandage so as to enable the user to affix the bandage by wrapping the bandage around the lip and/or alar sidewall of the nose (with one end being just inside of the lip or nose and the other end being adhesively attached to the skin) and applying gentle force to said malleable component to form it to the shape of the lip or nose.

In a second aspect, the present invention solves the problem of attachment of a bandage to the lip and/or area surrounding the lip and/or (alar sidewall of the) nose by incorporating a mucosal adhesive to that end of the adhesive bandage intended for inside of the mouth or nose so as to enable the user to adhesively attach said end to the inside of the mouth or nose and then attach the other end to the skin.

In a third aspect, the present invention solves the problem of attachment of a bandage to the lip and/or area surrounding the lips and/or (alar sidewall of the) nose by creation or incorporation of some combination of the aforementioned malleable component or material and mucosal adhesive.

In a forth aspect, the present invention solves the problem of attachment of a wound dressing to the lip and/or mouth area and/or nose of any non-human animal by using the spirit of the invention in an iteration suitable to the skin and mucosal tissue of the non-human animal.

In another aspect, the present invention relates to a cosmetic method for concealing wounds by using a combination of at least one of the above described lip and/or nose bandage iterations along with commercially available cosmetics (makeup) and stains of various lip tones. Such iteration comprises a base sheet fabricated of a material optimized for the acceptance of such cosmetics so as to camouflage the bandage itself.

In another aspect, the present invention relates to an inconspicuous bandage for concealing wounds by creating at least one of the above described lip and/or nose bandage iterations comprising a transparent base sheet and a dressing in one or more skin-toned colors.

In another aspect, the present invention relates to a kit containing several of the lip and/or nose bandages together with various medicaments appropriate for treating each stage of wound development/healing, for example, the blister stage, ulcer stage and scabbing stage of a herpetic cold sore, wherein said medicaments are either contained separately and applied to the bandage or wound by the user or alternatively, associated with the dressing during manufacturing so as to be transferred either immediately or over time from the dressing to the wound upon contact with the wound.

### BRIEF DESCRIPTION OF DRAWINGS

FIG. 1 is a perspective view of a human nose with a wound located on the exterior of the alar sidewall.

FIG. 2 is a perspective view of a human nose with a lip and/or nose bandage in accordance with the present invention secured to the alar sidewall.

FIG. 3 is a perspective view of a human mouth with two (2) lip and/or nose bandages in accordance with one embodiment of the present invention secured to the upper and lower lips.

FIG. 4 is a perspective view of a human mouth with two (2) lip and/or nose bandages in accordance with another embodiment of the present invention secured to the upper and lower lips.

FIG. 5 is a schematic perspective view showing the components of the lip and/or nose bandage according to a first embodiment of the present invention.

FIG. 6 is a schematic perspective view showing the components of the lip and/or nose bandage according to a second embodiment of the present invention.

FIG. 7 is a schematic perspective view showing the components of the lip and/or nose bandage according to a third embodiment of the present invention.

### DESCRIPTION OF EMBODIMENTS

As used herein, the term “wound” is intended to include all wounds (surgical or traumatic), scratches, sores, cold sores, blisters, fever blisters, ulcers, pimples, lesions, and burns.

As used herein, the term “bandage” is used interchangeably with “wound dressing” and refers to a covering to be placed over a wound.

Examples of embodiments of the invention are set forth below and reference is here made to each example. Examples are provided by way of explanation and not limitation of the invention. The spirit of the present invention is a bandage that will wrap and/or form and/or mold around the edge of the alar sidewall of the nose and/or around the upper or lower lip and remain affixed through a combination of the means herein referenced. It will be apparent to those of ordinary skill in the art that various modifications and variations can be made in the present invention without departing from the spirit and scope of the invention. It is intended that the present invention covers such modifications and variations as come within the scope of the appended claims and equivalents.

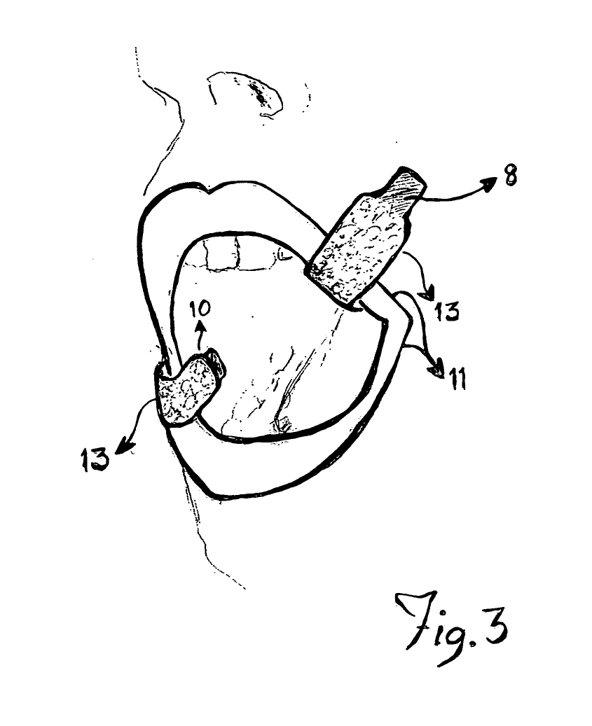

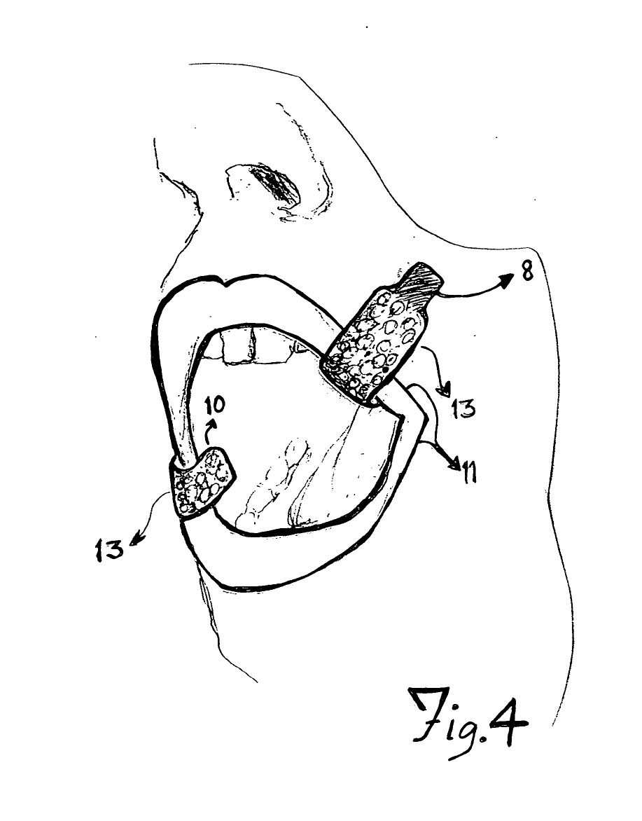

A lip and/or nose bandage 13 in accordance with the present invention is illustrated generally in FIGS. 2 and 3 and 4. In FIG. 2 a lip and/or nose bandage 13 is shown secured to a nose 6 and in FIGS. 3 and 4, to the upper and lower lips 11 of a wearer.

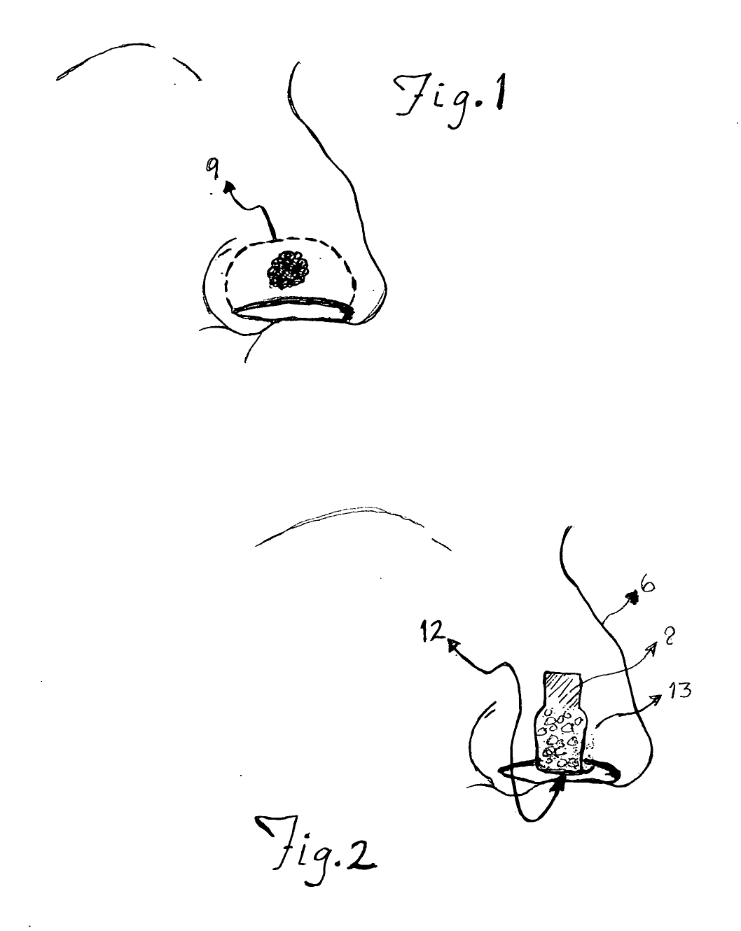

FIG. 1 shows a wound located on the exterior of the alar sidewall 9 of the nose. (It is also common for a wound to be similarly located on the interior of the alar sidewall of the nose.) FIG. 2 illustrates that in both circumstances, a lip and/or nose bandage can be used to cover both wound(s). This example shows a first end 8 {referred to hereafter as the external end (8)} conventionally adhesively attached to the skin. FIG. 2 further demonstrates an example of a lip and/or nose bandage 13 as it would appear on a user once applied, that is, wrapped 12 around the edge of the alar sidewall of the nose. FIGS. 3 and 4 show examples of a lip and/or nose bandage 13 as they would appear on a user once applied, that is, wrapped around the upper and/or lower lip(s) 11.

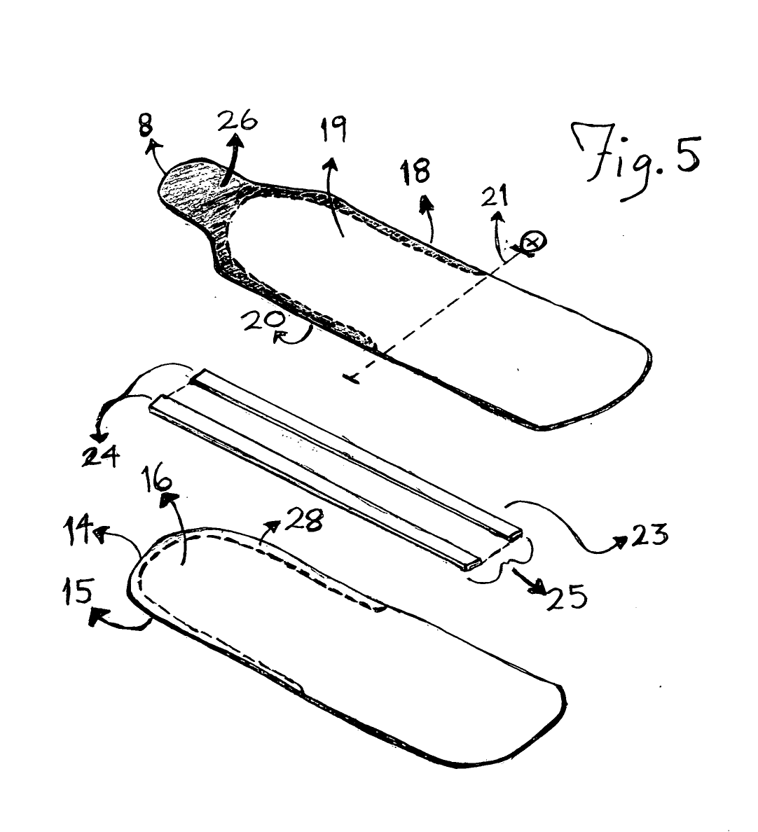

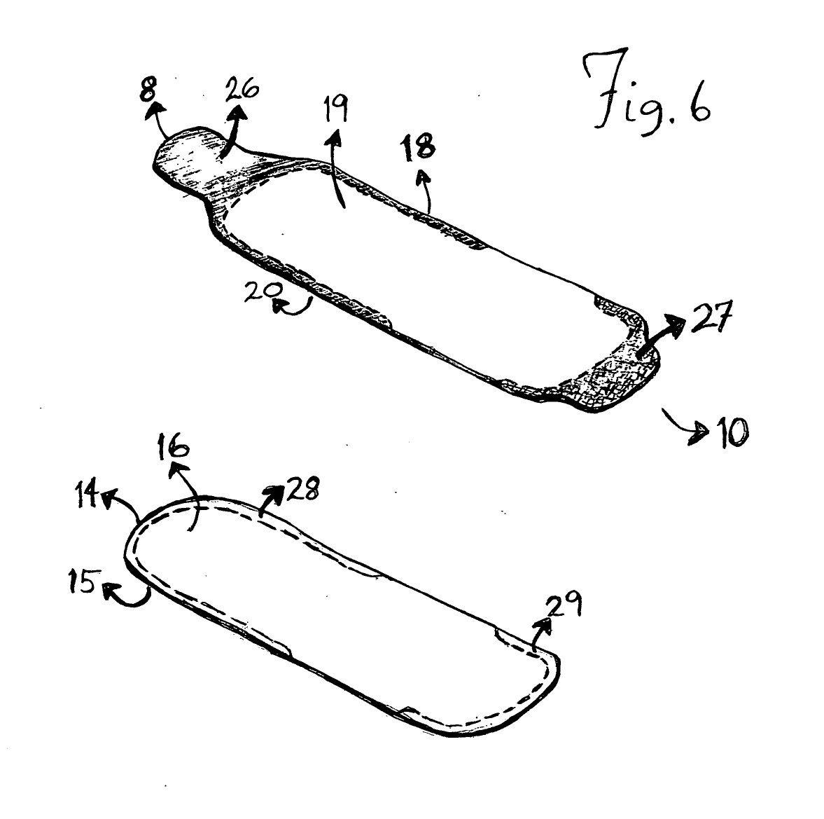

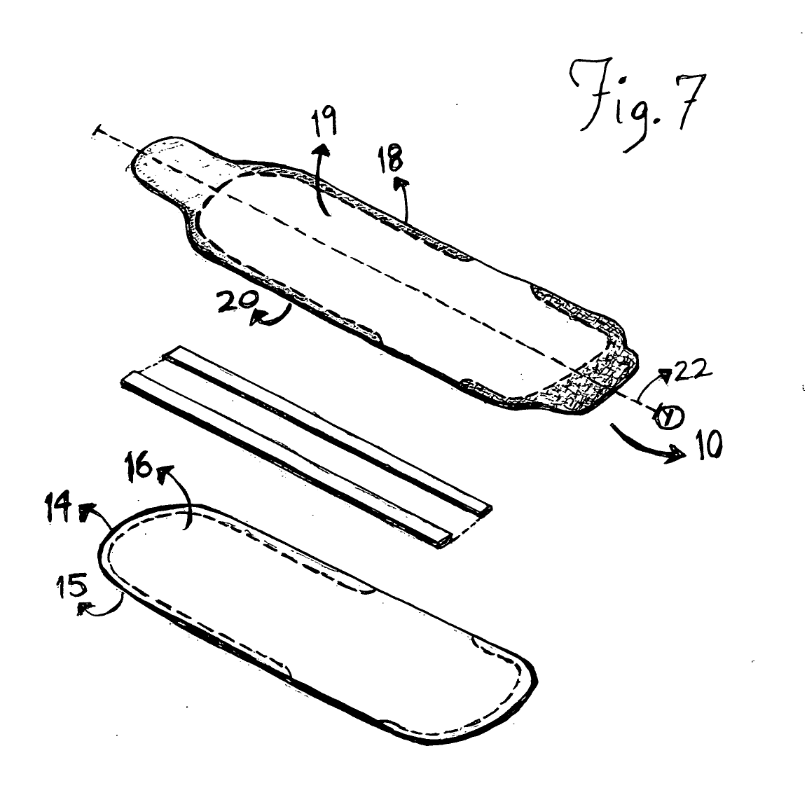

The lip and/or nose bandage can be made in many sizes to accommodate various sized wounds as is commonly practiced with production of adhesive bandages. In a preferred embodiment, as illustrated in FIG. 5, a lip and/or nose bandage measured at the named x-axis 21 can range in width between 7 and 30 mm and as illustrated in FIG. 7, in length (measured at the named y-axis 22) between 30 and 50 mm.

In FIGS. 2 and 3 and 4, the external end 8 of the bandage is adhered to the skin. The second end 10 {referred to hereafter as the internal end (10)} is affixed by at least one of three means; one means incorporates a malleable component (see FIG. 5), the second means incorporates a mucosal adhesive (see FIG. 6), and the third means is a combination of the malleable component and the mucosal adhesive (see FIG. 7). Reference now will be made in detail to these means.

As seen in FIGS. 5 and 6 and 7, all embodiments of the lip and/or nose bandage comprise a dressing 14 layer (that layer coming in contact with the wound) having a skin facing side 15 and a top side 16 and a base sheet 18 layer having a top surface 19 (that side seen by the user once applied) and a bottom surface 20. The dressing 14 can be made of many materials known to the art, for example, those which control moisture at the wound site or those which contain releasable medicaments and in all cases is desirably non-adherent, such as the HURT-FREE® dressing used in the Comfort Sheer BAND-AID® adhesive bandage. The base sheet 18 can be made of many materials known to the art, for example, those featuring higher levels of breathability and/or elastic properties. Given the multidirectional movement of that area surrounding the lips of a person engaged in speaking or eating, for example, the base sheet 18 is preferably elastic and highly flexible, such as the COMFORT-FLEX® base sheet used in the Comfort Sheer BAND-AID® adhesive bandage.

FIG. 5 shows a first embodiment of the present invention wherein a malleable component 23 is sandwiched between the dressing 14 and base sheet 18. The malleable component 23 can be made of any material capable of being easily wrapped and molded or formed into a desired shape, for example, by a thumb and forefinger, around a lip or alar sidewall and holding that shape. Additionally, the component is desirably produced from a material both economical and safe to use. The malleable component 23 can be incorporated as at least one strip 24, wire, or sheet 25 cut to a width and length appropriate to the width and length of the bandage. For example, three (3) lip and/or nose bandage prototypes (18 mm) in width were produced with favorable results using 1) an uncoated 0.5 mm metal wire, 2) a plastic-coated 1 mm metal wire, and 3) a 10 mm wide aluminum polylaminate. In a preferred embodiment, pure tin sheet (such as is known in the wine-making industry as the ideal material for bottle capsules) can be cut to occupy a surface area of at least 50% of the width and 60-70% of the length of the dressing. A bonding process such as that one used to sandwich plastic strips between the backing layer and the adhesive layer of Breathe Right® nasal strips may be used to bond the top side 16 of the dressing 14 to the bottom surface 20 of the base sheet 18 with the malleable component 23 sandwiched there between. A skin-friendly (biocompatible) adhesive, such as the high-tack adhesive Wacker Silpuran 2117, can be applied to all or some part of that area of the bottom surface 20 of the base sheet 18 demonstrated by the shaded section 26 on the external end 8 of the base sheet 18. That area 28 of the dressing 14 distinguished from the rest of the dressing by a dotted line may be cut away (as part of the manufacturing process) so as to match the area of application of skin adhesive on the bottom surface 20 of the base sheet 18.

FIG. 6 shows a second embodiment of the present invention wherein the means of attaching the internal end 10 of a lip and/or nose bandage is reliant upon a mucosal adhesive applied to all or some part of that area 27 of the bottom surface 20 of the base sheet 18 demonstrated by the shaded section on the internal end 10 of the base sheet 18. That area 29 of the dressing 14 distinguished from the rest of the dressing 14 by a dotted line may be cut away (as part of the manufacturing process) so as to match the area of application of mucosal adhesive on the bottom surface 20 of the base sheet 18. In a preferred embodiment, a biocompatible adhesive for use in an oral environment, such as is described in U.S. Pat. No. 9,012,530 can be used. A biocompatible adhesive, such as the high-tack adhesive Wacker Silpuran 2117, can be applied to all or some part of that area 26 of the bottom surface 20 of the base sheet 18 demonstrated by the shaded section on the external end 8 of the base sheet 18. That area 28 of the dressing 14 distinguished from the rest of the dressing by a dotted line may be cut away (as part of the manufacturing process) so as to match the area of application of skin adhesive on the bottom surface 20 of the base sheet 18.

FIG. 7 shows a third embodiment of the present invention wherein the features illustrated in the first and second embodiments of the present invention are combined, that is, as means to affix the internal end 10 of the lip and/or nose bandage.

### CITATION LIST

### Patent Literature

U.S. Pat. No. 1,111,679
U.S. Pat. No. 1,967,923
U.S. Pat. No. 2,226,546
U.S. Pat. No. 2,905,174
U.S. Pat. No. 3,696,811
U.S. Pat. No. 4,213,452
U.S. Pat. No. 4,274,402
U.S. Pat. No. 4,534,342
U.S. Pat. No. 5,762,494
U.S. Pat. No. 6,143,798
U.S. Pat. No. 6,211,243 B1
U.S. Pat. No. 6,309,500 B1
U.S. Pat. No. 6,318,362 B1
U.S. Pat. No. 6,967,261 B1
U.S. Pat. No. 7,943,811 B2
U.S. Pat. No. 9,012,530 B2

### Non Patent Literature

US 2006/0206047 A1
US 2014/0350526 A1
US 2015/0018743 A1
US 2015/0088085 A1
http://www.ashasexualhealt.ordstdsstisTherpes/fast-facts-and-faqs
http://www.ascounciLorg/blogpost/1268738/218868/Wacker-Launches-High-Tack-Silicone-Adhesive-for-Medical-Applications
http://www.g3enterprises.com/closures/capsules.asp
http://www.corksupplv.com/products/products-detail.aspx?pid=42&catid=31&returnurl=/products/capsules.aspx

I claim:

1. A bandage for use on the upper and/or lower lips and/or the alar sidewall of the nose of humans and other animals comprising:
a base sheet layer having a top surface (seen by the user once applied) and a bottom surface; and
a dressing layer; and
a release liner layer (not shown),
wherein the bandage remains affixed by a skin-friendly adhesive associated with a portion of the bottom surface of the base sheet on the exterior of the mouth or nose; and
wherein the bandage remains affixed on the interior of the mouth or nose by at least one (and possibly both) of the following means,
a malleable material layer (sandwiched between the base sheet and dressing); and/or
a mucosal adhesive associated with a portion of the bottom surface of the base sheet.

2. A bandage of claim 1 wherein the top surface of a base sheet is optimized for the acceptance of cosmetics (like makeup and lip stains) wherein a user may employ cosmetics to camouflage the bandage itself.

3. A bandage of claim 1 wherein a base sheet is transparent and a dressing is made of at least one skin-toned color.

4. A bandage of claim 1 wherein such bandage is offered as a kit containing several of the lip and/or nose bandages together with various medicaments appropriate for treating each stage or phase of wound development/healing, for example, the blister stage, ulcer stage and scabbing stage of a herpetic cold sore, wherein said medicaments are either contained separately and applied to the bandage or wound by the user or alternatively, associated with the dressing during manufacturing so as to be transferred either immediately or over time from the dressing to the wound upon contact with the wound.
